(19) United States
(12) Patent Application Publication
(10) Pub. No.: US 2012/0265264 A1
(43) Pub. Date: Oct. 18, 2012

(54) System and Method for Conditioning a Lithium Battery in an Automatic External Defibrillator

(75) Inventors: Gintaras A. Vaisnys, Chicago, IL (US); Giovanni C. Meier, Madison, CT (US); Glenn W. Laub, Princeton, NJ (US)

(73) Assignee: Defibtech, LLC, Guilford, CT (US)

(21) Appl. No.: 13/537,243

(22) Filed: Jun. 29, 2012

Related U.S. Application Data
(63) Continuation of application No. 12/057,504, filed on Mar. 28, 2008.

(51) Int. Cl. A61N 1/39

(52) U.S. Cl. 607/6

(57) ABSTRACT

An inventive system and method de-passivates a direct current (DC) power source of an Automatic External Defibrillator (AED), such as an AED lithium battery. The system includes a main processor and standby processor. The standby processor monitors the age and usage of the battery. Based on the status of the monitored parameters, the system executes a conditioning discharge to remove a layer of salt crystals on the DC power source.

### TECHNICAL FIELD

The present invention is generally directed to battery-powered cardiac automatic external defibrillator systems, and relates more particularly to the conditioning of a lithium battery in a portable automatic external defibrillator system.

### BACKGROUND OF THE INVENTION

Automatic external defibrillators (“AEDs”) are portable medical devices designed to supply a controlled electric shock to a person's heart during cardiac arrest. The electric shock stops fibrillations—fast, erratic contractions of the heart muscle that occur during cardiac arrest. Because the muscle contractions associated with fibrillations are unsynchronized, the heart is unable to circulate blood through the individual's body. As a result, death can result in a matter of minutes if the fibrillations are not treated quickly.

To provide rapid treatment for a person experiencing cardiac arrest, AEDs are designed to be operated by users with minimal training. Because AEDs can be used by non-medical personnel to treat sudden cardiac arrest (SCA), they are often deployed in a myriad of locations outside of traditional medical settings. As a result, more and more non-medical establishments are purchasing AEDs for deployment in their environments. AEDs are typically powered by stand alone battery systems.

AEDs are typically standby devices that are used infrequently and that remain in storage for long periods of time. This standby storage time can be on the order of months or even years. Minimizing power consumed by the AED while it is in standby mode during storage may extend the battery life of the system and reserve battery power for rescue attempts using the AED.

Because time is of the essence during a rescue attempt when a victim suffering from cardiac arrest is treated, AEDs are often deployed throughout large facilities (e.g., factories, office buildings, or large retail centers). Thus, regardless of where the victim is within the facility, access to an AED should only be minutes away. In large facilities, there may be many AEDs deployed throughout the facility.

Lithium batteries have a low rate of self-discharge, enabling them to deliver power even if they have been sitting idle for years. For this reason, lithium batteries are often optimal for powering portable AEDs. In part, the low rate of self-discharge for lithium batteries is made possible by introducing salt-forming organic compounds into the interior of the battery when they are manufactured. Over time, these compounds react to form a layer of salt crystals (i.e., a passivating layer) at the internal surfaces of the battery, including at the anode. The layer of salt crystals increases the internal resistance of the battery and helps to reduce the rate of self-discharge. In this way, a lithium battery with no applied load can last several years with no appreciable loss of charge.

However, if a lithium battery remains idle for an extended period of time, the layer of salt crystals can become so thick that it reduces the rate at which the battery can deliver power, even though the battery is still almost at full capacity (Le., nearly fully charged). This delay in power delivery can be on the order of several seconds. For many applications, this delay is not a problem. By applying a load to the battery, the layer of salt crystals can be driven off the internal surfaces of the battery and the battery can deliver power normally (i.e., without any passivation-induced delay).

For defibrillators such as AEDs, however, several problems are caused by the formation of the passivating layer. First, a self-test circuit that monitors battery life in an AED may erroneously conclude that the battery charge is low, when in fact the battery is merely passivated and contains sufficient charge to deliver a therapeutic shock. Another problem is that passivation-induced delays in power delivery of several seconds are unacceptably long for AEDs. Specifically, when an AED delivers a therapeutic electric shock to the heart of a person suffering from sudden cardiac arrest (SCA), the lithium battery of the AED must be capable of delivering power rapidly when called into service. Moreover, because sudden cardiac arrest is fatal within a matter of minutes if not properly treated, there may not be enough time to drive off the passivating layer of salt crystals by applying a load to the AED battery.

Accordingly, based on the foregoing there is a need for a method for reducing passivation of lithium batteries utilized to deliver a therapeutic shock in an AED.

### SUMMARY OF THE INVENTION

An inventive method and system can administer a conditioning discharge to drive off salt crystals from a defibrillator battery of a portable AED. The portable AED in the system may comprise a battery, main processor, and low-power processor. The low power processor may monitor when and how many self-tests have been performed by the defibrillator. The processor may also monitor how many defibrillation shocks have been performed by the defibrillator, as well as the age of the battery.

At periodic times, the processor of the portable AED can calculate whether a conditioning discharge should be applied based on the age and usage of the battery. For example, the processor may determine to perform a conditioning discharge as a function of the number of defibrillation shocks and self-tests performed by the AED battery. The frequency of conditioning discharges administered by the processor may increase with the age or usage of the battery.

In one exemplary embodiment, the processor of the portable AED may monitor the number of self-tests and defibrillation shocks administered by the defibrillator. Based on the number of these tasks, the processor is capable of applying a conditioning discharge to drive off salt crystals formed in the lithium battery. The conditioning discharge may comprise drawing a large amount of power from the AED battery, such as pulsing the battery at 150 Joules of energy, in order to drive off the layer of salt crystals formed within the battery.

### BRIEF DESCRIPTION OF THE DRAWINGS

FIG. 1 illustrates an automatic external defibrillator according to one exemplary embodiment of the invention.

FIG. 2 is a functional block diagram illustrating an AED according to one exemplary embodiment of the invention.

FIG. 3 is a logic flow diagram highlighting exemplary steps for executing a conditioning discharge to condition a lithium battery according to one exemplary embodiment of the invention.

FIG. 4 is a logic flow diagram illustrating exemplary steps for monitoring a self-test step according to one exemplary embodiment of the present invention.

FIG. 5 is a logic flow diagram illustrating exemplary steps for monitoring the number of defibrillation shocks administered by an AED according to one exemplary embodiment of the invention.

FIG. 6 is a logic flow diagram illustrating exemplary steps for determining if a conditional charge should be applied in an automatic external defibrillator according to one exemplary embodiment of the invention.

FIG. 7 is a logic flow diagram highlighting exemplary steps for executing a conditioning discharge in an AED according to one exemplary embodiment of the invention.

FIG. 8 is a logic flow diagram highlighting exemplary steps for executing a conditioning discharge in an AED according to one exemplary embodiment of the invention.

### DETAILED DESCRIPTION OF THE EXEMPLARY EMBODIMENTS

An inventive system and method can provide a conditioning discharge to drive off salt crystals from an automatic external defibrillator (AED) battery (i.e., in order to prevent the effects of passivation). An advantage of this inventive system is that it can counteract the effects of passivation without requiring any new hardware or physical modification of the battery. In this way, the inventive method is applicable to all commercially available batteries, and is especially suitable for lithium batteries.

One aspect of this invention is the realization that the rate of battery passivation increases as a function of several parameters, including the age of the battery and the remaining capacity of the battery. Thus the inventive method can utilize a progressive conditioning schedule or formula designed to combat the gradually increasing rate of battery passivation with battery age and repeated use. For AEDs, it should be noted that battery use is not limited to the delivery of a therapeutic electric shock to a victim of sudden cardiac arrest, but also includes periodic (e.g., daily) self-testing. Thus, the inventive method may also be advantageously applied to reduce passivation in an AED which has never been used to deliver a therapeutic shock, but has undergone repeated in-situ self-tests after deployment to an office building or some other facility.

In one exemplary embodiment, the inventive method provides a progressive schedule of conditioning discharges of the battery. A “conditioning discharge,” as used herein, refers to a battery discharge that is sufficient to drive off all (or a substantial amount) of the passivating layer of salt crystals within an AED battery. By way of example, a conditioning discharge may be a discharge of 150 Joules of energy, which may last from 6 to 10 seconds.

In preferred exemplary embodiments, the progressive schedule of conditioning discharges is controlled by a processor. The processor may be the built-in AED processor used for self-testing and/or delivery of a therapeutic shock, or it may be a separate, dedicated processor. In one exemplary embodiment, the processor keeps track of the number of times an AED has applied a load to the battery, either through the execution of its regularly scheduled self-test protocol or through the delivery of a therapeutic shock. Based on the number of times that a load has been applied, the processor may determine the frequency that a conditioning discharge should be applied. For example, if the AED battery is less than six months old, and no therapeutic electric shocks have been administered, the processor might require a conditioning discharge to occur periodically, for example, after every third monthly self test. On the other hand, if the battery is fifteen months old, the processor may require a conditioning discharge after every other monthly self-test in order to counteract the effects of the increasing rate of passivation.

In one exemplary embodiment, a processor may monitor the age of the AED battery and the usage of the battery (e.g., the number of defibrillation shocks administered by the AED battery and/or the number of self-tests performed by the AED). Based on this information, the processor may choose to initiate a conditioning discharge. This conditioning discharge may draw current from the AED battery for a short period of time in order to drive off one or more layers of salt-crystals that may have accumulated on the anode of the AED battery. In an exemplary embodiment, this conditioning discharge may be administered from 6 to 10 seconds at 2 amps in order to de-passivate the battery.

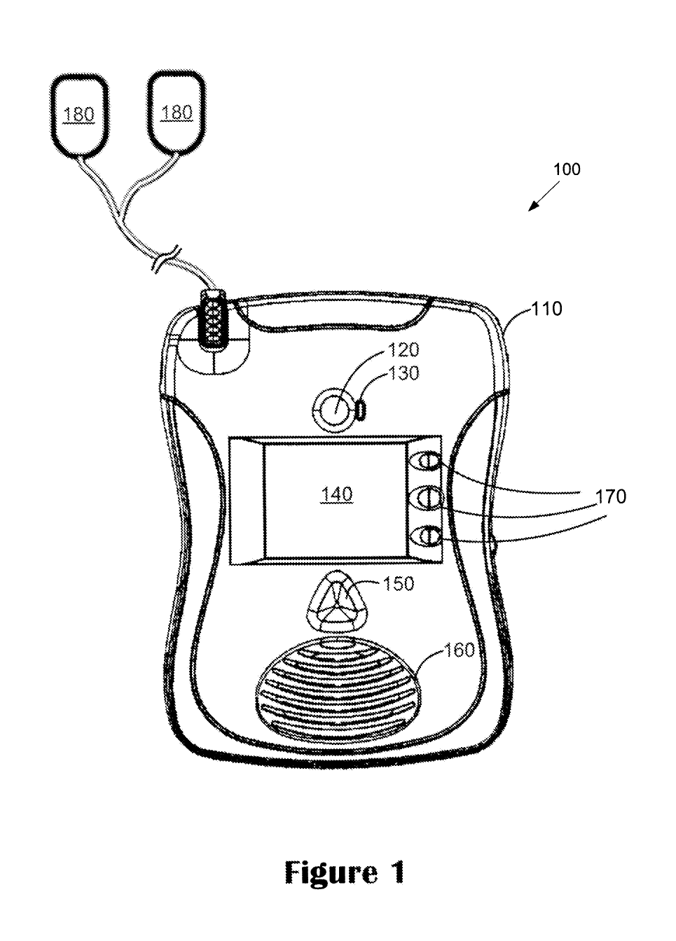

Turning now to the drawings, in which like reference numerals refer to like elements, FIG. 1 illustrates an AED 100 according to one exemplary embodiment of the invention. As illustrated, an AED 100 may comprise a casing 110 wherein an on/off button 120, video display 140, shock delivery actuator 150, and speaker 160 may be housed. Further, the AED may comprise a status indicator light 130 adjacent to the on/off button 120 and one or more buttons 170 adjacent to the video display 140. The buttons 170 may be used to enter information or program the AED (alternatively or additionally, the screen 140 may comprise a touch screen display for entering information into the AED 100).

The AED 100 may be capable of being connected to one or more defibrillation shock pads 180. In this way, the AED 100 may be used to administer a defibrillation shock when a person suffers a cardiac arrest. In particular, when the AED 100 is turned on, the speaker 160 may provide audible commands for initiating a shock using the shock delivery actuator 150. Additionally, the video display 140 may provide information on how to perform a defibrillation shock. For example, the video display 140 and speaker 160 may give directions on how the pads 180 should be applied to perform a defibrillation shock. When the pads 180 have been applied, the shock delivery actuator 150 may be depressed, sending an electrical charge through the defibrillation pads 180, and, in turn applying a shock to the person suffering from cardiac arrest.

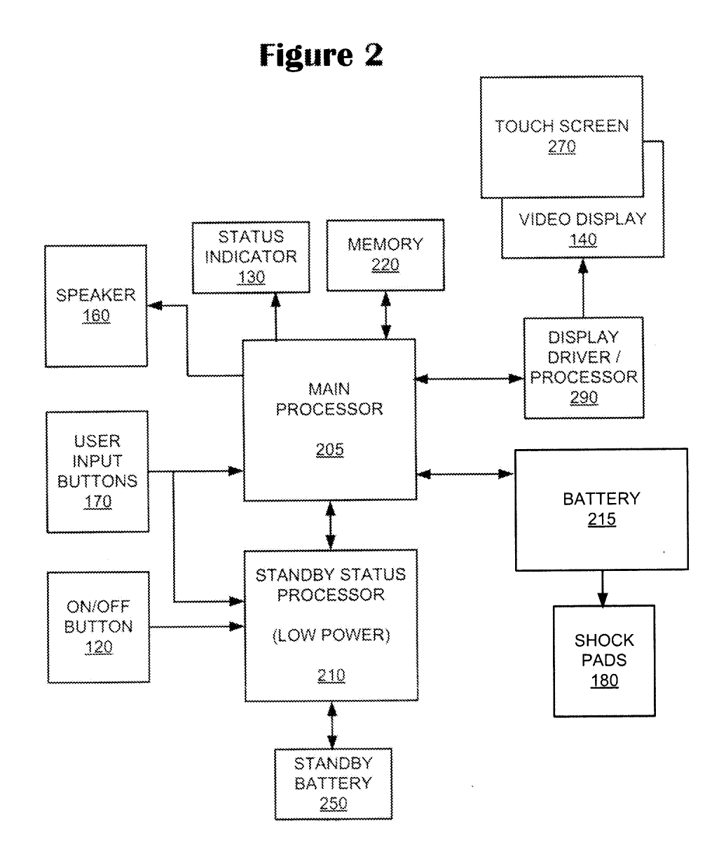

FIG. 2 is a functional block diagram illustrating an AED according to one exemplary embodiment of the invention. As illustrated, an AED may comprise a main processor 205 that is functionally connected to the on/off button 120, the speaker 160, a display/driver processor 290, an AED direct current battery 215, the status indicator light 130, memory 220, a standby processor 210, and the user input buttons 170. The display/driver processor 290 may be functionally connected to the video display 140, which may comprise a touch screen 270, and the AED battery 215 may be functionally connected to the shock pads 180. According to one exemplary embodiment, the AED battery 215 is employed to functionally deliver a defibrillation shock through the shock pads 180.

The standby processor 210 may be functionally connected to the user input buttons 170, the on/off button 120, and a standby direct current battery 250. The standby processor 210, which may spend most of the time in a low-power sleep mode, wakes every few seconds to sample sensors and actuate indicators (not shown). The standby processor 210 may also wake periodically to perform, or cause to be performed, built in self tests of the host system. The standby processor 210 may also monitor on/off button 120 in order to turn a main processor 205 on and off. According to an exemplary embodiment, the inventive methods described herein are controlled by the standby (or low power) processor 210. However, in alternative or additional exemplary embodiments, the main processor 205 may control whether or not a discharge is applied to condition the AED battery 215.

According to one exemplary embodiment, the standby processor 210 draws power and performs functions by utilizing the standby battery 250. In this manner, the standby battery 250 may provide energy to the standby processor 210 and/or main processor 205 in order to run self-tests and determine whether to perform a conditioning discharge. It is understood, however, that the main AED battery 215 may likewise be used to perform functions related to the standby processor, including, but not limited to, performing self-tests and determining whether to perform conditioning discharges.

One of ordinary skill in the art will appreciate that process functions or steps performed by the standby processor 210 may comprise firmware code executing on a microcontroller, microprocessor, or DSP processor; state machines implemented in application specific or programmable logic; or numerous other forms without departing from the spirit and scope of the invention. In other words, the invention may be provided as a computer program which may include a machine-readable medium having stored thereon instructions which may be used to program a computer (or other electronic devices) to perform a process according to the invention.

The machine-readable medium may include, but is not limited to, floppy diskettes, optical disks, CD-ROMs, and magneto-optical disks, ROMs, RAMs, EPROMs, EEPROMs, magnet or optical cards, flash memory, or other type of media/machine-readable medium suitable for storing electronic instructions.

Certain steps in the processes or process flow described in all of the logic flow diagrams referred to below must naturally precede others for the invention to function as described. However, the invention is not limited to the order of the steps described if such order or sequence does not alter the functionality of the present invention. That is, it is recognized that some steps may be performed before, after, or in parallel other steps without departing from the scope and spirit of the present invention. Additionally, it is recognized that certain steps could be re-arranged in different sequences or entirely deleted without deviating from the scope and spirit of the invention. In other words, it is recognized that the steps illustrated in the flow charts represent one way of achieving a desired result of determining whether to provide conditioning discharges for a battery of an AED. Other ways which may include additional, different steps or the elimination of steps, or the combination of eliminating steps and adding different steps will be apparent to one of ordinary skill in the art.

Further, one of ordinary skill in programming would be able to write such a computer program or identify the appropriate hardware circuits to implement the disclosed invention without difficulty based on the flow charts and associated description in the application text, for example. Therefore, disclosure of a particular set of program code instructions or detailed hardware devices is not considered necessary for an adequate understanding of how to make and use the invention. The inventive functionality of the claimed computer implemented processes will be explained in more detail in the following description in conjunction with the remaining Figures illustrating other process flows.

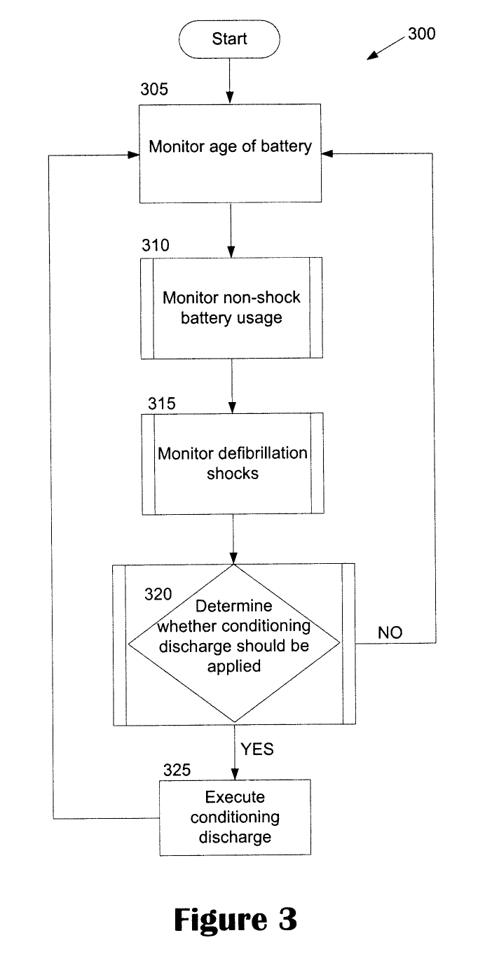

FIG. 3 is a logic flow diagram highlighting exemplary steps for determining whether to execute a conditioning discharge to condition a direct current power source, such as a lithium battery, according to one exemplary embodiment of the invention. Referring now to FIGS. 2 and 3, the exemplary method 300 begins at step 305 where a standby processor 210 monitors the age of the AED battery 215. This step may be performed by a calendar function that is kept in the AED memory 220. For example, when an AED battery 215 is placed in the AED 100, a timer may be started in the processor 210 that records the length of time until the next AED battery 215 is placed into the AED 100.

Continuing to routine 310, the standby processor 210 monitors the non-shock battery usage performed by the AED 100. One example of non-shock usage is a periodic self-test. A self-test is a procedure that can be performed at specified time intervals by the standby processor 210 to check the operability of the AED 100. For example, a self-test may check to ensure that the AED battery 215 is charged to a level sufficient to provide a defibrillation shock. If the self-test determines that the battery charge is insufficient, the standby processor 210 may notify a user of the problem by instructing the main processor 205 to flash the status indicator light 130 or provide a message on the video display 140.

Upon the occurrence of non-shock usage, the exemplary method in FIG. 3 increases a self-test counter in routine 310 and continues to routine 315, where the standby processor 210 monitors for the occurrence of defibrillation shocks. Routine 315 may be performed in a variety of ways; however, in one exemplary embodiment, the standby processor 210 may simply record the number of times the defibrillation shock is administered by the AED battery 215. Then, continuing to routine 320, the standby processor 210 may determine whether a conditioning discharge should be applied. An exemplary sub-method or sub-step will be described in more detail hereinafter; however, in general, the standby processor 210 may compare a conditioning schedule or formula to certain parameters, including, but not limited to, the age of the battery, the number of self-tests performed by the system, or the number of defibrillation shocks administered by the system, or any combination thereof.

Continuing the exemplary process illustrated in FIG. 3, if the standby processor 210 determines a discharge should be applied, then the “yes” path is followed to step 325, where the standby processor 210 executes a conditioning discharge. The conditioning discharge may be performed by the standby processor 210 instructing the main processor 205 to discharge the AED battery 215 for a specified period of time. According to an exemplary embodiment, the discharge must be sufficient to break down the layers of salt crystals that have formed on the anodes of the AED battery 215. This discharge may be 150 Joules, lasting from approximately 6 to 10 seconds.

Returning back to the routine 320, in the event that the standby processor 210 determines that a conditioning discharge should not be applied, then the “no” path is followed back to step 305, where the exemplary method illustrated in FIG. 3 repeats. Further, if a conditioning discharge is applied at step 325, the process also repeats by returning to step 305 once the conditioning discharge has been applied to the AED battery 215.

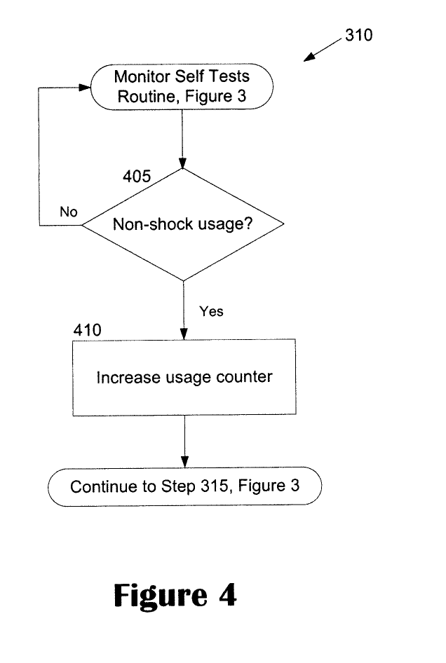

FIG. 4 is a logic flow diagram 400 illustrating exemplary steps for monitoring a self-test routine 310 according to one exemplary embodiment of the present invention. Referring to FIGS. 2 and 4, the exemplary routine 310 begins at step 405 by determining if the battery is being used for non-shock purposes. If non-shock usage occurs, then the “yes” path is followed to step 410, where the usage counter is increased by a value corresponding to the amount of energy used by the battery (e.g., the milliamps times seconds may be added to the usage counter). The usage counter may be maintained by the standby processor 210 in memory 220. If, at step 405, the battery is not being used, then the “no” path is followed and the standby processor continues monitoring battery usage. After the conditioning counter is increased by one at step 415, the exemplary method in FIG. 4 continues to routine 315, where the exemplary process illustrated in FIG. 3 continues.

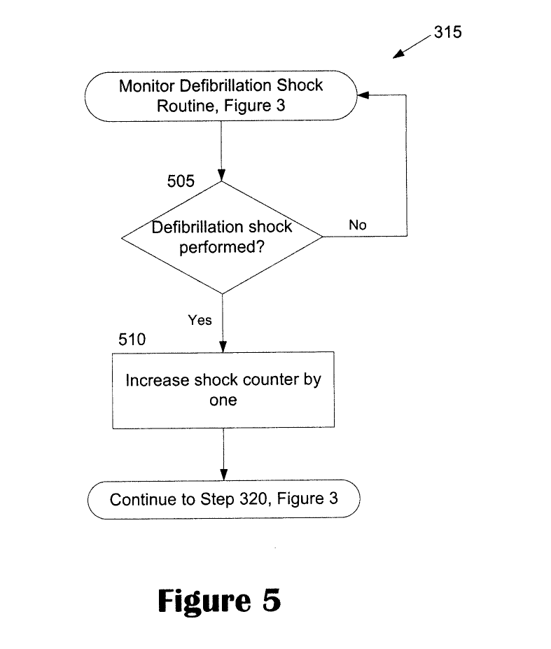

Referring now to FIG. 5, this figure is a logic flow diagram illustrating exemplary steps for monitoring the number of defibrillation shocks administered by an AED according to one exemplary embodiment of the invention. Referring to FIGS. 2 and 5, at step 505, the standby processor 210 monitors the AED 100 for the occurrence of a defibrillation shock. For instance, the standby processor may query the main processor 205 to determine if a defibrillation shock has been administered by the AED 100. If a defibrillation shock is detected, the process continues to step 515, where a shock counter is increased by one unit. This shock counter value may be maintained by the standby processor and stored in memory 220. Conversely, if a shock is not detected at step 510, then the “no” path is followed and the process may repeat.

As indicated, the standby processor 210 may monitor the main processor 205 for the execution of a defibrillation shock. A defibrillation shock may occur when the main AED battery 215 is engaged to supply a high voltage shock to someone suffering from cardiac arrest. According to an exemplary embodiment, at step 515, when the AED is used to administer a defibrillation shock, the shock counter is increased by one. In this way, the standby processor 210 is able to accurately track how many times a particular AED battery 215 has been used to administer a defibrillation shock. This data may then be used to determine whether a conditioning discharge is required, as will be discussed in more detail hereinafter.

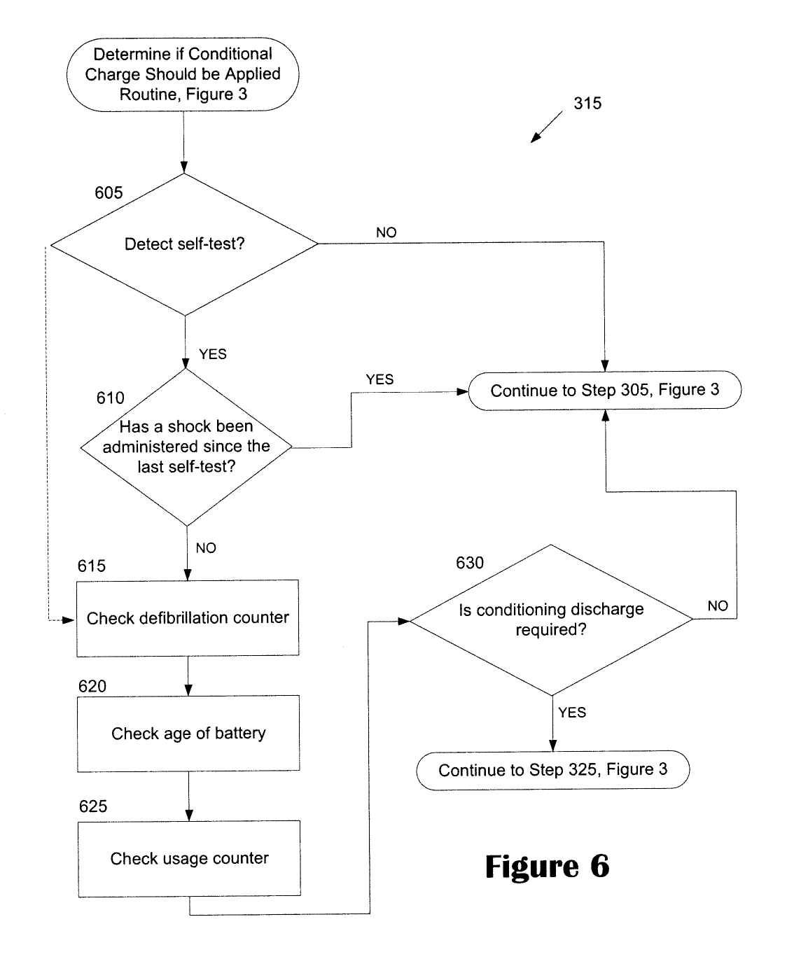

Referring now to FIG. 6, this figure is a logic flow diagram illustrating exemplary steps of routine 315 of FIG. 3 for determining if a conditional discharge should be applied in an AED 100 according to one exemplary embodiment of the invention. Referring to FIGS. 2 and 6, at routine 605, the standby processor 210 determines whether or not a self-test has been administered by the AED 100. If no self-test has been administered, and is not currently being administered, then the “no” path is followed to step 305, illustrated at FIG. 3, where the process repeats and waits for a self-test to begin. In this way, the AED 100 may determine whether to perform a conditioning discharge each time a self-test is performed; however, in alternative embodiments, the process may be performed independent of whether a self-test is or has occurred (e.g., the determination of whether a conditioning discharge should be applied may be made at other times, such as when the unit is turned on or off).

According to an exemplary embodiment, if a self-test is detected at routine 605, then the process follows the “yes” path to decision routine 610, where the standby processor 210 determines if the AED 100 has been used since the last self-test. If a defibrillation shock has been administered since the last self-test, then the process returns to step 305, as illustrated in FIG. 3, and awaits another self-test step. Thus, if a defibrillation shock has been performed since the last self-test, the exemplary process illustrated in FIG. 6 does not assess whether to provide a conditioning discharge. This is because a defibrillation shock performs a similar function to the conditioning discharge, thus alleviating the need for the inventive discharge process. Accordingly, if a defibrillation shock has been performed since the last self-test, the exemplary method 600 returns for the commencement of the next self-test to be performed. In the event that a defibrillation shock has not been administered since the last self-test, the process continues to step 615, where the standby processor 210 determines how many defibrillation shocks have been administered using the AED battery 215. Further, as illustrated, in certain alternative exemplary embodiments, step 610 may be by-passed entirely.

To determine how many shocks have been administered by the AED, the processor may access the defibrillation counter stored in memory 220 as discussed with reference to FIG. 5. Then, at step 620, the age of the battery may be checked by the standby processor 210. As discussed, this may be performed by examining the counter exhibiting how many days the AED battery 215 has been installed in the defibrillation device. Once the age of the AED direct-current power source, such as battery 215, has been assessed, the process continues to step 625, where the usage counter is checked by the processor 210. As discussed with reference to FIG. 4, the usage counter can be stored in memory 220 and can be increased, for example, upon each subsequent self-test administered by the standby processor 210.

After the standby processor 210 has obtained the defibrillation shock count, the age of the battery, and the self-test counter information, it can compare the information to a pre-defined schedule or formula in decision step 630. A schedule or formula may be stored in memory 220. By accessing the schedule or formula with reference to the collected parameters, the standby processor 210 is able to assess whether or not a conditioning discharge should be applied to the AED battery 215. An exemplary schedule or formula may be used to apply a conditioning discharge based on various parameters of the AED. In one exemplary embodiment, the usage counter value may be converted to an equivalent shock count and added to the shock counter value to form a single “Counter” value and compared to a table to determine whether a conditioning discharge is required. An exemplary schedule for assessing whether to discharge the battery is illustrated in Table 1 below.

TABLE 1
Exemplary schedule for assessing whether to perform conditioning
discharge.

| Counter Number | Administer Shock? |
|---|---|
| Counter <15 | Every Third Counter Increase |
| Counter >= 15-30 | Every Second Counter Increase |
| Counter >30 | Every Counter Increase |

As illustrated in Table 1, the frequency of conditioning discharges increases as a function of the age and usage of the battery. This reflects the principal that the greater the number of discharges (e.g., usage) from the battery, the more likely passivation will accumulate on the AED battery, thus requiring a conditioning discharge. Thus, the frequency of the conditioning discharge increases over the life of the battery. Accordingly, a battery that is less than a year old (i.e., less than 364 days old) will receive fewer conditioning discharges than a battery that is two years old.

Continuing through the exemplary process illustrated in FIG. 6, if a conditioning discharge is required at decision step 630 (based on the above formula or an alternative formula or table stored in memory), then the “yes” path is followed back to step 325, illustrated in FIG. 3, where the process repeats. However, if a conditioning discharge is not required at step 630, then the process continues and returns to step 305, as illustrated in FIG. 3. Further, as illustrated in FIG. 6, if a defibrillation shock has been administered since the last self-test, the exemplary process may (in certain exemplary embodiments) automatically forego administering a conditioning discharge. This is because a defibrillation shock is typically sufficient to de-passivate a battery, and, therefore, the AED would not require a conditioning discharge.

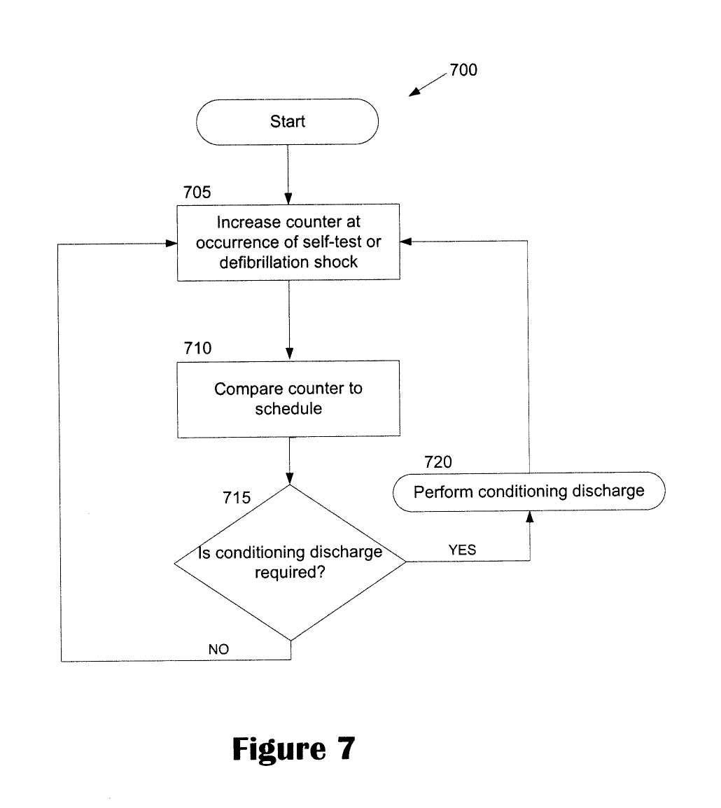

FIG. 7 is a logic flow diagram highlighting exemplary steps for executing a conditioning discharge in an AED according to another exemplary embodiment of the inventive system. At step 705, a counter is increased upon the occurrence of a defibrillation shock or monthly self-test. Then, at step 710, the counter may be compared to a schedule, such as the one shown at Table 1. In step 715, the processor can determine whether a conditioning discharge is required, based on the comparison of the counter value to the schedule. Hence, based on this comparison, the AED will either perform a conditioning discharge at step 720 or the return to step 705 to repeat the exemplary routine 700.

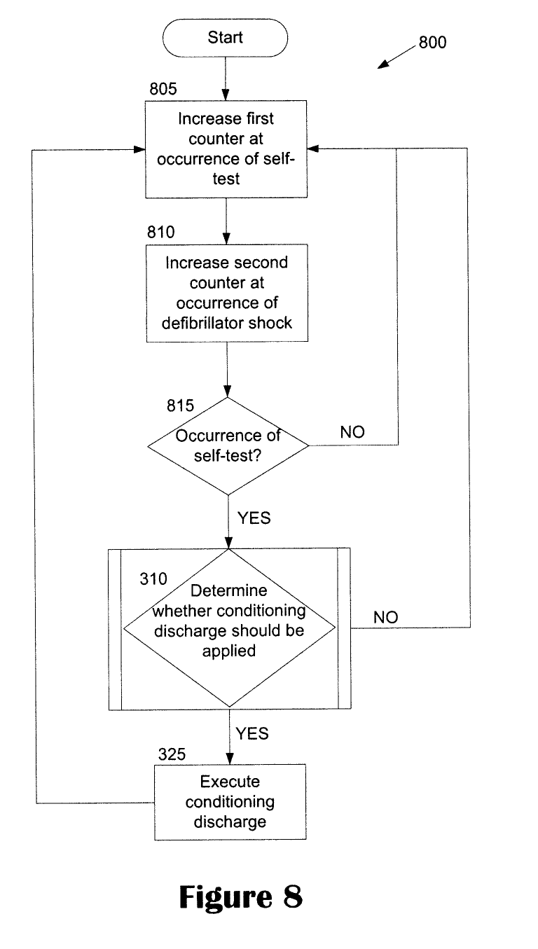

FIG. 8 is a logic flow diagram highlighting exemplary steps for executing a conditioning discharge in an AED according to another exemplary embodiment of the inventive system. As illustrated, the process 800 begins at step 805, where a first counter is increased at the occurrence of a self-test. According to an exemplary embodiment, a standby processor 210 maintains a counter in memory that increases each time a self-test is performed by the AED 100. Continuing in the exemplary process, at step 810, a second counter is increased at the occurrence of a defibrillation shock. Similar to the first counter, in an exemplary embodiment, the second counter may be stored in memory and incremented by a standby processor 210 in the AED 100. Accordingly, the second counter may be increased by one unit when the AED 100 administers a defibrillation shock to a person suffering from cardiac arrest.

At decision step 815, the standby processor 210 determines whether or not a self-test is presently occurring. If a self-test is occurring, then the exemplary method continues to decision routine 310, as illustrated with reference to FIG. 3. However, if a self-test is not occurring, then the processor returns back to step 805, where the exemplary method repeats.

As previously discussed, at decision routine 310 the standby processor 210 assesses the age of the battery, the number of defibrillation shocks administered by the battery, and the number of self-tests performed since the last conditioning discharge has been applied to determine if a conditioning discharge should be applied. In a typical conditioning discharge, the AED battery 215 releases 150 Joules of energy in order to drive off the layer of salt crystals that may have collected around the anodes of the battery 215. This process may take anywhere from 6 to 10 seconds at 2 amps to successfully remove the layer of salt crystals. For example, in one exemplary embodiment, the standby processor 210 may instruct the main processor 205 to draw current from the AED battery 215 for two seconds to de-passivate the battery 215.

While the system and method of the inventive system and method have been described in exemplary embodiments, alternative embodiments of an AED system and method will be come apparent to one of ordinary skill in the art to which the present invention pertains without departing from its spirit and scope. Therefore, although this invention has been described in exemplary form with a certain degree of particularity, it should be understood that the present disclosure has been made only by way of example, and the numerous changes and the details of construction and the combination and arrangement of parts or steps may be resorted to without departing from the spirit of scope of the invention. Accordingly, the scope of the present invention is defined by the appended claims rather than the foregoing description.

1-23. (canceled)

24. A system for driving off a passivation layer from a direct current (DC) power source of a portable automatic external defibrillator (AED), the system comprising:
the DC power source disposed in the portable AED;
a main processor disposed in the portable AED and coupled to the DC power source; and
a standby processor disposed in the portable AED, wherein the standby processor performs the steps of:
monitoring a number of times the portable AED has entered a self-test mode;
determining if there is a need to depassivate the DC power source, such determination based on the number of times the self-test mode has been entered; and
if a depassivation need is determined, instructing the main processor to draw current from the DC power source to drive off salt crystals collected on the DC power source.

25. The system of claim 24, wherein the standby processor further performs the step of monitoring an age of the DC power source, and in the step of determining if there is a need to depassivate the DC power source, the determination is additionally based on the age of the DC power source.

26. The system of claim 24, wherein the standby processor further performs the step of monitoring a number of times the portable AED has been used to administer a defibrillation shock, and in the step of determining if there is a need to depassivate the DC power source, the determination is additionally based on the number of times the portable AED has been used to administer a defibrillation shock.

27. The system of claim 24, wherein the standby processor instructs the main processor to draw current from the DC power source based on the age of the battery.

28. The system of claim 24, wherein the main processor performs the step of drawing current from the DC power source for an amount of time sufficient to drive off a layer of salt crystals within the DC power source.

29. The system of claim 28, wherein the main processor performs the step of drawing current from the DC power source for at least six seconds at 2 amps.

30. The system of claim 28, wherein the main processor discharges 150 Joules of energy from the DC power source based on the instruction from the standby processor.

31. The system of claim 24, wherein the DC power source is a battery.

32. The system of claim 31, wherein the battery is a lithium battery.

33. A system for driving off a passivation layer from a direct current (DC) power source of a portable automatic external defibrillator (AED), the system comprising:
the DC power source disposed in the portable AED;
a main processor disposed in the portable AED and coupled to the DC power source; and
a standby processor disposed in the portable AED, wherein the standby processor performs the steps of:
monitoring a number of times the portable AED has been used to administer a defibrillation shock;
determining if there is a need to depassivate the DC power source, such determination based on the number of times a defibrillation shock has been given; and
if a depassivation need is determined, instructing the main processor to draw current from the DC power source to drive off salt crystals collected on the DC power source.

34. The system of claim 33, wherein the standby processor further performs the step of monitoring an age of the DC power source, and in the step of determining if there is a need to depassivate the DC power source, the determination is additionally based on the age.

35. The system of claim 33, wherein the standby processor further performs the step of monitoring a number of times the portable AED has entered a self-test mode, and in the step of determining if there is a need to depassivate the DC power source, the determination is additionally based on the number of times the portable AED has entered the self-test mode.

36. A system for driving off a passivation layer from a direct current (DC) power source of a portable automatic external defibrillator (AED), the system comprising:
the DC power source disposed in the portable AED;
a main processor disposed in the portable AED and coupled to the DC power source; and
a standby processor disposed in the portable AED, wherein the standby processor performs the steps of:
monitoring an age of the DC power source;
determining if there is a need to depassivate the DC power source, such determination based on the age of the DC power source; and
if a depassivation need is determined, instructing the main processor to draw current from the DC power source to drive off salt crystals collected on the DC power source.

37. The system of claim 36, wherein the standby processor further performs the step of monitoring a number of times the self-test mode has been entered, and in the step of determining if there is a need to depassivate the DC power source, the determination is additionally based on the number of times the self-test mode has been entered.

38. The system of claim 36, wherein the standby processor further performs the step of monitoring a number of times the portable AED has been used to administer a defibrillation shock, and in the step of determining if there is a need to depassivate the DC power source, the determination is additionally based on the number of times the portable AED has been used to administer a defibrillation shock.
